(19) United States
(12) Patent Application Publication
(10) Pub. No.: US 2006/0046961 A1
(43) Pub. Date: Mar. 2, 2006

(54) Controlled and directed local delivery of anti-inflammatory compositions

(76) Inventors: William F. McKay, Memphis, TN (US); John M. Zanella, Cordova, TN (US)

Correspondence Address: ELMORE PATENT LAW GROUP, PC, 209 MAIN STREET, N. CHELMSFORD, MA, 01863, US

(21) Appl. No.: 11/091,348

(22) Filed: Mar. 28, 2005

Related U.S. Application Data
(63) Continuation-in-part of application No. 10/932,878, filed on Sep. 2, 2004.

(51) Int. Cl. A61K 38/17

(52) U.S. Cl. 514/12

(57) ABSTRACT

The invention provides a method for alleviating pain associated with neuromuscular or skeletal injury or inflammation by controlled and directed delivery of one or more biological response modifiers to inhibit the inflammatory response which ultimately causes acute or chronic pain. Controlled and directed delivery can be provided by implantable or infusion pumps, implantable controlled release devices, or by sustained release compositions comprising biological response modifiers.

### FIELD OF THE INVENTION

The present invention relates to systems and methods for decreasing or eliminating pain, particularly when associated with musculoskeletal disease, injury or surgery. More specifically, the invention relates to methods for administering biological response modifiers to inhibit or eliminate the inflammatory response that may result in acute or chronic pain.

### BACKGROUND OF THE INVENTION

Tumor necrosis factor alpha (TNF-α) appears early in the inflammatory cascade following infection or injury. It is produced by monocytes, macrophages, and T lymphocytes. TNF-α exerts its primary effects on monocytes, synovial macrophages, fibroblasts, chondrocytes, and endothelial cells, and stimulates proinflammatory cytokine and chemokine synthesis. It activates granulocytes, and increases MHC Class II expression. It promotes secretion of matrix metalloproteinases (MMPs), leading to cartilage matrix degradation. Because it initiates an inflammatory cascade, and has been found to be increased in close proximity to inflamed or injured tissue, TNF-α inhibition is a target for pain therapy. Pro-TNF-α is expressed on the plasma membrane, then cleaved in the extracellular domain. Trimerization is required for biological activity. TNF-α acts through two receptors (TNFRs): Type I receptors (p60, p55, CD 120a) are expressed constitutively on most cell types and Type II receptors (p80, p75, CD 120b) are inducible. Popular TNF-α inhibitors act primarily to inhibit binding of TNF-α to its receptors. There are currently two major classes of TNF inhibitors: 1) monoclonal antibodies to TNF-α, which prevent binding of TNF-α to its two cell-associated signaling receptors (p55 and p75) and 2) monomeric soluble forms of p55 or p75 TNFR dimerized by linking them to an immunoglobulin (Ig) Fc fragment. These Igs bind to TNF-α with high affinity and prevent it from binding to its cell-associated receptor.

TNF inhibitors have therefore been developed for therapeutic use for orthopedic and neuromuscular disease or injury that can cause pain, such as rheumatoid arthritis. TNF inhibitors currently in use are generally administered systemically via intravenous infusion and subcutaneous injection, but there are side effects of anti-TNF therapies associated with the higher doses and systemic administration that are common with these therapies. In the case of direct injection, a bolus of the pharmaceutical agent is injected as near to the target site as placement of a needle will allow. Unfortunately, it provides a limited quantity of agent that must move through the tissue to the target site. This method is inadequate to serve the needs of patients. Anti-TNF therapy is generally needed over an extended period of time, so repeated injections are likely to be necessary. Injection site pain and reactions sometimes develop with anti-TNF agents.

What is needed is a system and method for controlled and directed delivery of biological response modifier, such as TNF inhibitors, for the treatment and prevention of inflammation and pain, capable of being delivered for an extended period of time at, or in close proximity to, a targeted site such as the site of trauma or inflammation.

### SUMMARY OF THE INVENTION

The present invention relates to methods and systems for reducing pain and/or inflammation, a method for reducing pain, the method comprising administering to a target site in a subject in need of treatment an effective amount of a pharmaceutical composition comprising one or more biological response modifiers (BRM), wherein the one or more biological response modifiers are administered by a controlled administration system. In the practice of the invention, the administration is localized and sustained. For example, the administration occurs over a period of from about at least one day to about three months. In one embodiment the administration is continuous. The administration may also be periodic.

The pharmaceutical composition employed in the invention has a targeted release rate. For example, the targeted release rate is from about 24 hours to about 31 days. In another embodiment the targeted release rate is from about at least one day to about three months.

In the practice of the invention, the controlled administration system is implanted in a subject at or near a target site. Non-limiting examples of such sites include but are not limited to an inflamed nerve or a spinal site, in particular a spinal disc site. In one embodiment, the controlled administration system is conveniently a depot. In other embodiments, the controlled administration system is an infusion pump, an osmotic pump and/or an interbody pump. In the practice of the invention a depot is contained within any of the above listed pumps.

In one method of the invention, the controlled administration system comprises a system administered locally by insertion of a catheter at or near a target site, the catheter having a proximal end and a distal end, the proximal end having an opening to deliver a pharmaceutical in situ, the distal end being fluidly connected to a pharmaceutical delivery pump. For example, the proximal end of the catheter delivers the biological response modifier within 10 cm of the target site, more particularly, within 5 cm of the target site.

In certain embodiments, the biological response modifier of the invention inhibits inflammation mediated by TNF-α for example when the biological response modifier is a TNF-α receptor inhibitor. Suitable biological response modifiers include but are not limited to soluble tumor necrosis factor α receptors, pegylated soluble tumor necrosis factor α receptors, monoclonal antibodies, polyclonal antibodies, antibody fragments, COX-2 inhibitors, metalloprotease inhibitors, glutamate antagonists, glial cell derived neurotrophic factors, B2 receptor antagonists, Substance P receptor (NK1) antagonists, Downstream regulatory element antagonistic modulator (DREAM), iNOS, inhibitors of tetrodotoxin (TTX)-resistant Na+-channel receptor subtypes PN3 and SNS2, inhibitors of interleukins, TNF binding protein, dominant-negative TNF variants, Nanobodies™, kinase inhibitors, and combinations thereof. Other suitable biological response modifiers include but are not limited to Adalimumab, Infliximab, Etanercept, Pegsunercept (PEG sTNF-R1), Onercept, Kineret®, sTNF-R1, CDP-870, CDP-571, CNI-1493, RDP58, ISIS 104838, 1→3-β-D-glucans, Lenercept, PEG-sTNFRII Fc Mutein, D2E7, Afelimomab, AMG 108, 6-methoxy-2-napthylacetic acid) or betamethasone, capsaiein, civanide, TNFRc, ISIS2302 and GI 129471, integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, Tocilizumab, Chugai), HMGB-1 mAb (Critical Therapeutics Inc.), anti-IL2R antibody (daclizumab, basilicimab), ABX (anti IL-8 antibody), recombinant human IL-1 0, HuMax IL-15 (anti-IL 15 antibody) and combinations thereof.

In certain embodiments, the biological response modifier is administered in conjunction with an osteoinductive factor. Suitable osteoinductive factors include but are not limited to a bone morphogenetic protein or biologically active fragment or variant thereof, a LIM mineralization protein or biologically active fragment or variant thereof, or combinations thereof.

The invention also includes an implant comprising a pharmaceutical composition comprising one or more biopolymers and at least one biological response modifier. For example the biopolymers include but are not limited to poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, polyphosphoesters, polyanhydrides, polyester-anhydrides, polyamino acids, polyurethane-esters, polyphosphazines, polycaprolactones, polytrimethylene carbonates, polydioxanones, polyamide-esters, polyketals, polyacetals, glycosaminoglycans, hyaluronic acid, hyaluronic acid esters, polyethylene-vinyl acetates, silicones, polyurethanes, polypropylene fumarates, polydesaminotyrosine carbonates, polydesaminotyrosine arylates, polydesaminotyrosine ester carbonates, polydesamnotyrosine ester arylates, polyethylene oxides, polyorthocarbonates, polycarbonates, or copolymers or physical blends thereof or combinations thereof. In one embodiment, the biological response modifier is selected from the group consisting of soluble tumor necrosis factor α receptors, pegylated soluble tumor necrosis factor α receptors, monoclonal antibodies, polyclonal antibodies, antibody fragments and combinations thereof.

In the employment of the implant of the invention the biological response modifier includes but is not limited to Adalimumab, Infliximab, Etanercept, Pegsunercept (PEG sTNF-R1), sTNF-R1, CDP-870, CDP-571, CNI-1493, RDP58, ISIS 104838, 1→3-β-D-glucans, Remicade, Lenercept, PEG-sTNFRII Fc Mutein, D2E7, Afelimomab, and combinations thereof.

Also disclosed in that the one or more biological response modifiers are incorporated into a sustained release pharmaceutical composition. In one embodiment, two or more biological response modifiers are incorporated into a sustained release pharmaceutical composition. For example, in one embodiment two or more biological response modifiers are separately incorporated into separate biocompatible polymers.

The inventions also includes a method for treating osteolysis and/or bone resorption comprising administering to an osteolytic site in a subject in need of treatment an effective amount of a pharmaceutical composition comprising one or more biological response modifiers, wherein administration of the pharmaceutical composition is localized and sustained.

In one embodiment, the one or more biological response modifiers is administered in conjunction with at least one osteoinductive factor. Examples of suitable osteoinductive factor includes a bone morphogenetic protein or a biologically active fragment thereof, a LIM mineralization protein or a biologically active fragment thereof, or combinations thereof.

In yet another embodiment, a method for alleviating pain associated with bone tumors, the method comprising administering to a tumor site in a subject in need of treatment an effective amount of a composition comprising one or more biological response modifiers, wherein administration of the composition is localized and sustained. In this method the one or more biological response modifiers is administered in conjunction with at least one osteoinductive factor. Suitable examples include but are not limited to a bone morphogenetic protein or biologically active fragment or variant thereof, a LIM mineralization protein or biologically active fragment or variant thereof, or combinations thereof.

Also provided is a system for providing pain relief medication in a mammalian subject, the system comprising controlled administration system for providing controlled and directed delivery of at least one biological response modifier to a target site in a subject in need thereof comprising an effective amount of a composition comprising at least one biological response modifier which decreases inflammation at the target site. In another embodiment, the biological response modifier further comprises a modified release pharmaceutical composition. In yet another embodiment, the controlled administration system is a depot. The system can further comprising two or more biological response modifiers. In some systems, the controlled administration system is an osmotic pump or an interbody pump. In still another embodiment, the controlled administration system comprises a catheter having a proximal end and a distal end, the proximal end having an opening to deliver a pharmaceutical in situ, the distal end being fluidly connected to a pharmaceutical pump. In another embodiment, the proximal end of the catheter delivers the biological response modifier within about 10 cm of or closer to the target site. In another embodiment, the catheter delivers the biological response modifier within about 5 cm of or closer to the target site. In this system, the at least one biological response modifier inhibits inflammation mediated by TNF-α. Suitable examples of a biological response modifier is a TNF-α receptor inhibitor, for example, pegylated soluble TNF-α receptor. Other suitable biological response modifiers are listed herein. The system further comprises a therapeutically effective amount of at least one osteoinductive factor. Suitable osteoinductive factors include but are not limited to a bone morphogenetic protein or biologically active fragment or variant thereof, a LIM mineralization protein or biologically active fragment or variant thereof, or combinations thereof. In one embodiment, they system of the invention employs a depot comprising a modified release pharmaceutical carrier.

The invention also includes the use of a composition comprising one or more biological response modifiers which decrease inflammation at a target site for the manufacture of a pharmaceutical for reducing pain, wherein administration of an effective amount of the composition to a target site in a subject in need of treatment is localized and controlled. In the practice of this invention, the administration of the composition to a target site in a subject in need of treatment is localized and controlled.

In one embodiment, the invention is a controlled administration system for alleviating pain and limiting bone loss associated with osteolysis, wherein the administration of the composition to an osteolytic site in a subject in need of treatment is localized and controlled.

In another embodiment the invention includes the use of a composition comprising one or more biological response modifiers which decrease inflammation at a target site for the manufacture of a pharmaceutical for alleviating pain associated with bone tumors, wherein administration of the composition to a tumor site in a subject in need of treatment is localized and controlled.

In any of the above listed uses, the composition is a sustained release pharmaceutical composition.

Additional biological response modifiers are suitable for the methods, compositions and uses described herein. Such biological response modifiers include but are not intended to be limited to a COX-2 inhibitor, such as 6-methoxy-2-napthylacetic acid) or betamethasone or a metalloprotease inhibitor such as TAPI. Other biological response modifiers is selected from the group consisting of glutamate antagonists, glial cell-derived neurotropic factors (GDNF), B2 receptor antagonists, Substance P receptor (NK1) antagonists, Downstream regulatory element antagonistic modulator (DREAM), iNOS, inhibitors of tetrodotoxin (TTX)-resistant Na+-channel receptor subtypes PN3 and SNS2, inhibitors of interleukins. In one embodiment, the Substance P receptor (NK1) antagonists is capsaicin or civanide. In another embodiment, the inhibitor of interleukin is selected from the group consisting of IL-1, IL-6 IL-8, and IL-10. Further suitable biological response modifiers, include a TNF binding protein, for example, Onercept. Still another suitable biological response modifier includes a kinase inhibitor such as but not limited to Gleevec, Herceptin, Iressa, imatinib (STI571), herbimycin A, tyrphostin 47, erbstatin, genistein, staurosporine, PD98059, SB203580, CNI-1493, VX-50/702, SB203580, BIRB 796, Glaxo P38 MAP Kinase inhibitor, RWJ67657, UO126, Gd, SCIO-469, RO3201195, and Semipimod. Still other suitable biological response modifiers include ISIS2302, GI 129471, integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, Tocilizumab, Chugai), HMGB-1 mAb (Critical Therapeutics Inc.), anti-IL2R antibody (daclizumab, basilicimab), ABX (anti IL-8 antibody), recombinant human IL-10, HuMax IL-15 (anti-IL 15 antibody).

Also disclosed in a method for retarding tissue necrosis and/or damage, the method comprising administering to a target site in a subject in need of treatment an effective amount of a pharmaceutical composition comprising one or more biological response modifiers, wherein the one or more biological response modifiers are administered by controlled administration system which system is localized and sustained. In one embodiment, the controlled administration system is implanted in a subject at or near a target site such as but not limited to an inflamed nerve or a spinal site, for example into a spinal disc or spinal disc space.

### BRIEF DESCRIPTION OF THE DRAWINGS

FIG. 1ais an illustration of one embodiment of the invention comprising an interbody pump 1 for dispensing in vivo pharmaceutical compositions 2 through a catheter 3 to a location in situ near an inflammatory site (labeled as number 4).

FIG. 1bis an illustration of another embodiment of the invention comprising an interbody pump 1 for in vivo dispensing pharmaceutical compositions 2 through a catheter 3 within the inflammatory site 4 itself.

FIG. 2ais an illustration of another embodiment of the invention comprising an implant 5 containing pharmaceutical composition 6 placed within an inflammatory site 4.

FIG. 2bis an illustration of another embodiment of the invention comprising an implant 5 containing pharmaceutical composition 6 placed at an in situ location near an inflammatory site 4.

FIG. 3 is a graph demonstration the cumulative elution of Etanercept (Enbrel®) from PGLA microspheres over time (in days).

FIG. 4 is a graph demonstration the cumulative elution of Etanercept (Enbrel®) from PGLA millicynlinders (three different rods) over time (in days).

FIG. 5 is a bar graph representing data from the Paw Withdrawal Latency Test which measures hyperalgesia as described in the Examples.

FIG. 6 is a bar graph representing data from the Von Frey Testing which measures mechanical tactile allodynia as described in the Examples.

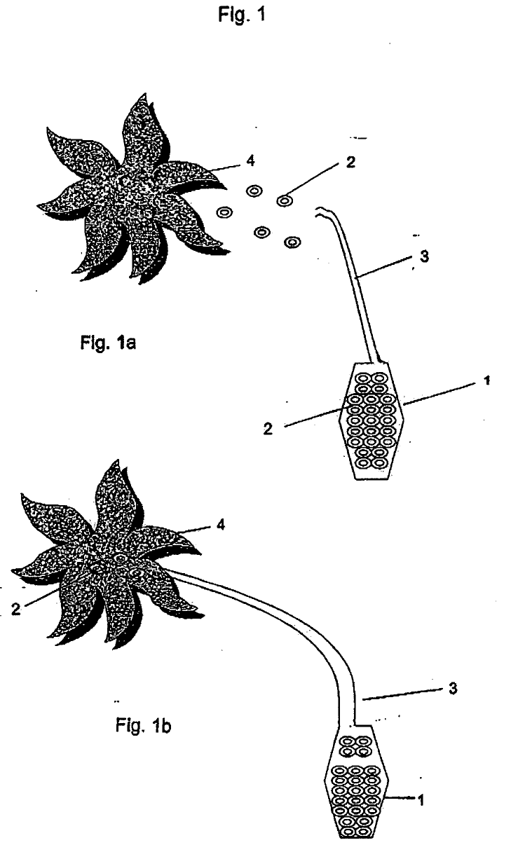

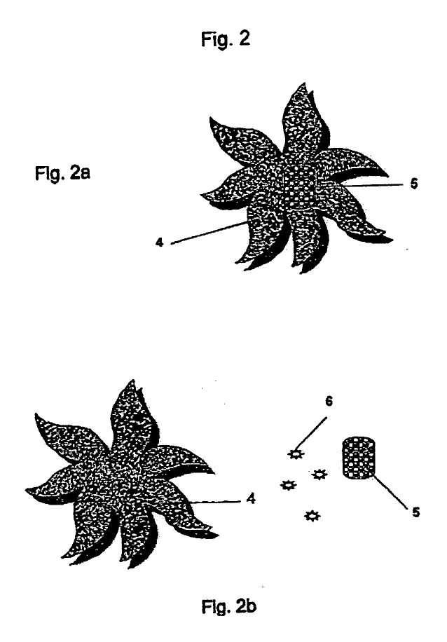

### DETAILED DESCRIPTION

The inventors provide systems and methods for decreasing, eliminating, or managing pain—especially pain of neuromuscular or skeletal origin—by providing direct and controlled delivery of at least one biological response modifier to one or more sites of inflammation and sources of pain. A biological response modifier itself may be on a continuum of rapid acting to long acting. Generally, the biological response modifier is a component of a pharmaceutical composition which can range in a continuum of rapid release to sustained release. Still further, the delivery of that pharmaceutical composition via the controlled administration system of the invention can include, for example, rapid and repeating delivery at intervals or continuous delivery. The delivery can be “local”, “direct” and “controlled.”

As used herein, biological response modifiers (BRMs) are substances that are direct and local-acting modulators of the pro-inflammatory effect of TNF-α such as but not limited to, for example, soluble tumor necrosis factor α receptors, any pegylated soluble tumor necrosis factor a receptor, monoclonal or polyclonal antibodies or antibody fragments or combinations thereof. Suitable examples include but are not limited to Adalimumab, Infliximab, Etanercept, Pegsunercept (PEG sTNF-R1), sTNF-R1, CDP-870, CDP-571, CNI-1493, RDP58, ISIS 104838, 1→3-β-D-glucans, Lenercept, PEG-sTNFRII Fc Mutein, D2E7, Afelimomab, and combinations thereof. They can decrease pain through their actions as inhibitors or agonists of the release of pro-inflammatory molecules. For example, these substances can act by inhibiting or antagonizing expression or binding of cytokines or other molecules that act in the early inflammatory cascade, often resulting in the downstream release of prostaglandins and leukotrienes. These substances can also act, for example, by blocking or antagonizing the binding of excitatory molecules to nociceptive receptors in the nervous system or neuromuscular system, as these receptors often trigger an inflammatory response to inflammation or injury of the nerve or surrounding tissue through a nitric oxide-mediated mechanism. These biological response modifiers include, for example, inhibitors of the action of tumor necrosis factor alpha (TNF-α). Studies have demonstrated that in chronic arthritic diseases, for example, cartilage degradation continues even when the inflammation has been suppressed. Biological response modifiers such as anti-TNF agents are particularly effective for joint pain, for example, because they not only decrease the inflammation that provides the source of pain but also slow the progression of joint destruction that may accompany the inflammatory response. Hence, local targeted delivery of the BRMs in accordance with the invention reduces tissue necrosis and damage.

Inflammation can be an acute response to trauma or a chronic response to the presence of inflammatory agents. When tissues are damaged, TNF-α attaches to cells to cause them to release other cytokines that cause inflammation. The purpose of the inflammatory cascade is to promote healing of the damaged tissue, but once the tissue is healed the inflammatory process does not necessarily end. Left unchecked, this can lead to degradation of surrounding tissues and associated chronic pain. Thus, pain can become a disease state in itself. That is, when this pathway is activated, inflammation and pain ensue. Often a vicious and seemingly endless cycle of insult, inflammation, and pain sets in. Examples of conditions in which this cycle is present includes, but is not limited to, rheumatoid arthritis, osteoarthritis, carpal tunnel syndrome, lower back pain, lower extremity pain, upper extremity, tissue pain and pain associated with injury or repair of cervical, thoracic and/or lumbar vertebrae or intervertebral discs, rotator cuff, articular joint, TMJ, tendons, ligaments and muscles.

It is understood that TNF is both affected by upstream events which modulate its production and, in turn, affects downstream events. Alternative approaches to treating the conditions exploit this known fact and BRMs are designed to specifically target TNF as well as molecules upstream, downstream and/or a combination thereof. Such approaches include, but are not limited to modulating TNF directly, modulating kinases, inhibiting cell-signaling, manipulating second messenger systems, modulating kinase activation signals, modulating a cluster designator on an inflammatory cell, modulating other receptors on inflammatory cells, blocking transcription or translation of TNF or other targets in pathway, modulating TNF-α post-translational effects, employing gene silencing, modulating interleukins, for example IL-1, IL-6 and IL-8. As used herein, “modulating” ranges from initiating to shutting down, and within that range is included enhancing significantly or slightly to inhibiting significantly or slightly. The term “inhibiting” includes a downregulation which may reduce or eliminate the targeted function, such as the production of a protein or the translation of an oligonucleotide sequence. For example, a given patient's condition may require only inhibition of a single molecule, such as TNF, or modulating more than one molecule in cascade of upstream and/or downstream events in the pathway.

In certain embodiments, TNF-α inhibitors reduce chronic discogenic back and leg pain if delivered by perispinal administration.

In other embodiments, a BRM is a COX2 inhibitor. Cyclooxygenase inhibitor is a class of enzymes that are believed to regulate the synthesis of prostaglandin E2 (PGE2). PGE2 may increase discogenic back pain by inducing radioculopathy. Inhibiting COX enzymes serves to reduce low back pain. While not intending to be bound to a single theory, it is believed that since they are regulators of PGE2s, they reduce low back pain by decreasing PGE2 production. One suitable COX2 inhibitor (6-methoxy-2-napthylacetic acid) has been shown to suppress PGE2 production and local inflammation in cell culture As decribed by Melarange et al. (1992a), Anti-inflammatory and gastroinstestinal effects of nabumetone or its active metabolite, 6MNA (6-methoxy-2-na[hthylacetic acid): comparison with indomethacin. Agents Actions., Spec No: C82-83; and (1992b) Antiinflammatory and gastrointestinal effects of nabumetone or its active metabolite, 6-methoxy-2-naphthylacetic acid (6MNA). Comparative studies with indomethacin, Dig Dis Sci., 37(12): 1847-1852. Another PGE2 inhibitor includes betamethasone.

Another suitable BRM is a metalloprotease inhibitor. For example, TAPI, is a metalloprotease inhibitor which can block cleavage of TNF-α which, in turn, will reduce production of TNF-α.

Still other suitable BRMs include: Glutamate antagonists, glial cell-derived neurotropic factors (GDNF), B₂ receptor antagonists, Substance P receptor (NK1) antagonists such as capsaicin and civanide, Downstream regulatory element antagonistic modulator (DREAM), iNOS, inhibitors of tetrodotoxin (TTX)-resistant Na⁺-channel receptor subtypes PN3 and SNS2, inhibitors of interleukins such as IL-1, IL-6 and IL-8, and anti-inflammatory cytokines such as IL-10.

In one example of an alternative approach, the BRM is a TNF binding protein. One suitable such BRM is currently referred to as Onercept. Formulae including Onercept, Onercept-like agents, and derivatives are all considered acceptable. Still other suitable BRMs include dominant-negative TNF variants. A suitable dominant-negative TNF variant includes but is not limited to DN-TNF and including those described by Steed et al. (2003), “Inactivation of TNF signaling by rationally designed dominant-negative TNF variants,” Science, 301(5641):1895-1898. Still more embodiments include the use of recombinant adeno-associated viral (rAAV) vector technology platform to deliver the oligonucleotides encoding inhibitors, enhancers, potentiators, neutralizers, or other modifiers. For example, in one embodiment (rAAV) vector technology platform to deliver the DNA sequence a potent inhibitor of tumor necrosis factor (TNF-alpha). One suitable inhibitor is TNFR:Fc. Other BRM include antibodies, including but not limited to naturally occurring or synthetic, double chain, single chained, or fragments thereof. For example, suitable BRM include molecules are based on single chain antibodies called Nanobodies™ (Ablynx, Ghent Belgium) which are defined as the smallest functional fragment of a naturally-occurring single domain antibody.

Alternatively, therapies to inhibit kinases and/or inhibit cell signaling are employed. Therapies that fall in this category are capable of manipulating the second messenger systems. Kinase activation signals multiple downstream effectors including those involving phosphatidylinositol 3-kinase and mitogen-activated protein kinases (MAPK), p38 MAPK, Src and protein tyrosine kinase (PTK). One example includes the signaling of TNFα effects is the downstream activation of MAPK.

Examples of kinase inhibitors are Gleevec, Herceptin, Iressa, imatinib (STI571), herbimycin A, tyrphostin 47, erbstatin, genistein, staurosporine, PD98059, SB203580, CNI-1493, VX-50/702 (Vertex/Kissei), SB203580, BIRB 796 (Boehringer Ingelheim), Glaxo P38 MAP Kinase inhibitor, RWJ67657 (J&J), UO126, Gd, SCIO-469 (Scios), RO3201195 (Roche), Semipimod (Cytokine PharmaSciences) or derivatives of the above mentioned agents. Yet another embodiment of the invention provides for the use of BRM which block the transcription or translation of TNF-α or other proteins in the inflammation cascade in acute pain.

BRMs which inhibit TNF-α-post translational effects are useful in the invention. For example, the initiation of TNF-α signaling cascade results in the enhanced production of numerous factors that subsequently act in a paracrine and autocrine fashion to elicit further production of TNF-α as well as other pro-inflammatory agents (IL-1, IL-6, IL-8, HMG-B1). Extracellular TNF-α modifying BRMs that act on the signals downstream of TNF-α are useful in treating systemic inflammatory diseases. Some of these BRMs are designed to block other effector molecules while others block the cellular interaction needed to further induce their production, for example, integrins and cell adhesion molecules.

Suitable BRMs include: integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, Tocilizumab, Chugai), HMGB-1 mAb (Critical Therapeutics Inc.), anti-IL2R antibody (daclizumab, basilicimab), ABX (anti IL-8 antibody), recombinant human IL-10, HuMax IL-15 (anti-IL 15 antibody).

Other suitable BRMs include IL-1 inhibitors. Interleukin-1 is a pro-inflammatory cytokine similar in action to TNF-α. For example, certain inhibitors of this protein are similar to those developed inhibit TNF-α. One such example is Kineret® (anakinra) which is a recombinant, non-glycosylated form of the human inerleukin-1 receptor antagonist (IL-1Ra). Another suitable BRM is AMG 108 which is a monoclonal antibody that blocks the action of IL-1.

As mentioned above, pain can become a disease state in itself. One particular area in which this is particularly true is in the lower back and legs. For example, disk herniation is a major cause of back pain and sciatica. Sciatica, or radiculopathy, is pain that radiates down the back of the legs and is generally thought to be caused by irritation of the roots of the sciatic nerve. Back pain can also be caused by spinal stenosis, characterized by overgrowth of bony or soft tissue in the spinal canal with associated pressure on the adjacent nerves. Degeneration of the facet joints between the vertebrae, tumors, infections, fractures, and inflammation of surrounding soft tissues can also cause back pain.

Forces that damage the vertebrae can injure the spinal cord through stretching, laceration, ischemia, or compression. Cancer can metastasize to the spine, resulting in bone destruction and spinal cord compression. Prolonged, continuous pressure on an extremity can result in a crush injury. Prior spine surgery, accompanied by the presence of spinal hardware, makes the spine stiff and vulnerable to additional injury. In all these situations, there is an inflammatory response to the injury. This response can become the source of significant, and often chronic, pain. It is this response that the present invention addresses by providing at least one inhibitor of an activator of the response. The inhibitor or combination of inhibitors is provided at, or in close proximity to, the source of inflammation, and is provided in a sustained dosage that is readily available for delivery at regular intervals, continuously, or as needed to manage the inflammatory response. This dosage can be provided, for example, by means of a controlled administration system.

As used herein a “controlled administration system” is a direct and local administration system to deliver biological response modifiers and includes, but is not limited to, a depot, an osmotic pump, an interbody pump, infusion pump, implantable mini-pumps, a peristaltic pump, other pharmaceutical pumps, or a system administered locally by insertion of a catheter at or near a target site, the catheter being operably connected to a pharmaceutical delivery pump. It is understood that pumps can be internal or external as appropriate. A “depot” includes but is not limited to capsules, microspheres, particles, gels, coating, matrices, wafers, pills or other pharmaceutical delivery compositions. A depot may comprise a biopolymer. The biopolymer may provide for non-immediate release. Examples of suitable sustained release biopolymers include but are not limited to poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, or combinations thereof.

In certain embodiments, the dosage is provided by means of a depot, a pharmaceutical pump or through a sustained delivery device implanted to provide the dosage at, or in close proximity to, the inflammatory site.

The ability to deliver pharmaceutical compositions comprising biological response modifiers in effective amounts directly to the site of trauma and/or inflammation is problematic in certain respects. As used herein, a pharmaceutical composition comprises at least one biological response modifier, alone or as part of, on, with, within or complexed with a depot and optionally diluents, excipients and other pharmaceutically acceptable agents desirable for improved stability, manufacturing, efficacy and the like.

It is desirable that controlled administration system be able to accurately, precisely and reliably deliver the intended amount of drug over the intended period of time. Many BRMs are quite expensive, especially those formulated to retain stability and efficacy over extended periods of time. Thus, the ability to efficiently formulate, process, package and deliver the BRM delivered via the controlled administration system with minimal loss of drug stability and efficacy is desirable. It is desirable that the pharmaceutical compositions suitable for controlled administration systems of the instant invention be carefully formulated for the desired modulation of inflammation in a controlled, local and direct manner. Among the features of the invention is the flexibility of the dosing option made possible due to the unique combinations of the controlled administration system(s) and the pharmaceutical compositions. The drug itself may be on a continuum of rapid acting to long acting. Further, the pharmaceutical composition itself can range in a continuum of rapid release or sustained release. Still further, the options for delivery of that pharmaceutical composition is on a continuum and includes but is not limited to rapid and repeating delivery at intervals ranging to continuous delivery. Delivery may occur at a desired site over a desired period of time for adequate distribution and absorption in the patient. Advantageously, when the controlled administration system is implanted, the delivery is capable of be directed to sites which are deep, complicated, painful or dangerous to reach by conventional means and/or otherwise inaccessible. As used throughout, the term “a” is intended to include the singular as well as plural.

In one embodiment, the invention provides localized delivery in a controlled manner, such as that provided by the controlled release system of the invention. In such an embodiment, the continued up and down cycling of biological response modifier levels in the patient can be avoided, allowing the body to adjust more easily to the level of the biological response modifier. Side effects can therefore be minimized.

The controlled administration system of the invention includes, for example, an infusion pump that administers a pharmaceutical composition through a catheter near the spine or one or more inflamed joints, an implantable mini-pump that can be inserted at an inflammatory site or site of injury or surgery, an implantable controlled release device (such as, for example, the device described in U.S. Pat. No. 6,001,386), and a sustained release delivery system (such as the system described in U.S. Pat. No. 6,007,843).

The pharmaceutical composition can also be administered in a controlled and sustained manner by implanting the desired one or more biological response modifiers dispersed within a depot such as polymer matrix that breaks down over time within the tissues, or otherwise incorporated within a protective coating that provides for the delay of the release of the one or more biological response modifiers.

One example of a suitable pump is the SynchroMed® (Medtronic, Minneapolis, Minnesota) pump. This pump has three sealed chambers. One contains an electronic module and battery. The second contains a peristaltic pump and drug reservoir. The third contains an inert gas, which provides the pressure needed to force the pharmaceutical composition into the peristaltic pump. To fill the pump, the pharmaceutical composition is injected through the reservoir fill port to the expandable reservoir. The inert gas creates pressure on the reservoir, and the pressure forces the pharmaceutical composition through a filter and into the pump chamber. The pharmaceutical composition is then pumped out of the device from the pump chamber and into the catheter, which will direct it for deposit at the target site. The rate of delivery of pharmaceutical composition is controlled by a microprocessor. This allows the pump to be used to deliver similar or different amounts of pharmaceutical composition continuously, at specific times, or at set intervals between deliveries.

Potential drug delivery devices suitable for adaptation for the method of the invention include but are not limited those described, for example, in U.S. Pat. No. 6,551,290 (Elsberry, et al.), which describes a medical catheter for target specific drug delivery; U.S. Pat. No. 6,571,125 (Thompson), which describes an implantable medical device for controllably releasing a biologically-active agent; U.S. Pat. No. 6,594,880 (Elsberry), which describes an intraparenchymal infusion catheter system for delivering therapeutic agents to selected sites in an organism; and U.S. Pat. No. 5,752,930 (Rise, et al.), which describes an implantable catheter for infusing equal volumes of agents to spaced sites.

Additional designs which may be adapted to be employed in the method of the present invention are provided, for example, in U.S. Pat. applications such as US 2002/0082583 (a pre-programmable implantable apparatus with a feedback regulated delivery method), US 2004/0106914 (a micro-reservoir osmotic release system for controlled release of chemicals), US 2004/0064088 (a small, light-weight device for delivering liquid medication), US 2004/0082908 (an implantable microminiature infusion device), US 2004/0098113 (an implantable ceramic valve pump assembly), and US 2004/0065615 (an implantable infusion pump with a collapsible fluid chamber). Alzet® osmotic pumps (Durect Corporation, Cupertino, Calif.) are also available in a variety of sizes, pumping rates and durations suitable for use in the method of the present invention.

Suitable polymers for use in the method of the present invention can comprise, for example, poly(alpha-hydroxy acids) such as poly(lactide-co-glycolide) (PLGA), polylactide (PLA), polyglycolide (PG), as well as polyethylene glycol (PEG) conjugates thereof. Polyorthoesters, polyaspirins, polyphosphagenes, and hydrogel materials such as collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, and PLGA-PEO-PLGA are also suitable. The polymers may be employed in the preparation of extended-release or sustained release compositions for use in the method of the present invention.

In one embodiment, further excipients are employed. The amount of excipient that is useful in the composition of this invention is an amount that serves to uniformly distribute the active agent throughout the composition so that it can be uniformly dispersed when it is to be delivered to a subject in need thereof. It may serve to dilute the biological response modifier to a concentration at which the BRM can provide the desired beneficial palliative or curative results while at the same time minimizing any adverse side effects that might occur from too high a concentration. It may also have a preservative effect. Thus, for a BRM that has high physiological activity, more of the excipient will be employed. On the other hand, for a BRM that exhibits a lower physiological activity a lesser quantity of the excipient will be employed. In general, the amount of excipient in the composition will be between about 50% weight (w) and 99.9% w. of the total composition. For BRM that have a particularly high physiological activity, the amount will be between about 98.0% and about 99.9% w.

Examples of suitable biological response modifiers include receptor antagonists, molecules that compete with the receptor for binding to the target molecule, antisense polynucleotides, and inhibitors of transcription of the DNA encoding the target protein. TNF-α antagonists may, for example, include Adalimumab, Infliximab, Etanercept, CNI-1493 (an inhibitor of macrophage activation and TNF-α release), RDP58 (Rationally Designed Peptide—a small molecule developed by SangStat Medical (Genzyme, Cambridge, Mass.) that inhibits TNF-α synthesis by preventing translation of TNF-α mRNA), and ISIS 104838 (an antisense TNF-α inhibitor). Still other suitable BRM include, any pegylated soluble tumor necrosis factor alpha receptor, for example, sTNF-R1, CDP-870, CDP-571, 1→3-β-D-glucans, Lenercept, PEG-sTNFRII Fc Mutein, D2E7, Afelimomab, Pegsunercept, other monoclonal or polyclonal antibodies or antibody fragments or mixtures thereof.

Natural compounds may also decrease TNF-α mRNA expression and can be delivered in controlled release form or by implantable or external controlled administration systems to inhibit expression of TNF-α to decrease or inhibit pain, for example, pain caused by the inflammatory cascade initiated by TNF-α. TNF-α inhibitors can act by inhibiting TNF-α transcription, translation, or receptor binding or activation, for example.

Excitatory amino acids such as glutamate and aspartate have been shown to play a role in the development of pain originating from nerves. Mice with blocked glutamate receptors, for example, have been shown to have a reduction in their responses to pain. Glutamate binds to two major classes of receptors: inotropic glutamate receptors (ligand-gated ion channels) and metabotropic receptors (G protein-coupled receptors). The inotropic receptors in the spinal cord include the N-methyl-D-aspartic acid (NMDA) receptors, the α-amino-3-hydroxy-5-methyl-4-isoxazolepropionate (AMPA) receptors, and the kainite receptors. In the method of the present invention, one or more biological response modifiers can include, for example, antagonists or inhibitors of glutamate binding to NMDA receptors, AMPA receptors, and/or kainate receptors.

Interleukin-1 receptor antagonists, thalidomide (a TNF-α release inhibitor), thalidomide analogues (which reduce TNF-α production by macrophages), bone morphogenetic protein (BMP) type 2 and BMP-4 (inhibitors of caspase 8, a TNF-α activator), quinapril (an inhibitor of angiotensin II, which upregulates TNF-α), interferons such as IL-11 (which modulate TNF-α receptor expression), and aurin-tricarboxylic acid (which inhibits TNF-α), for example, may also be useful for reducing inflammation-associated pain when provided in the method of the present invention. It is contemplated that where desirable a pegylated form of the above may be used.

Delivery of biological response modifiers to decrease or eliminate pain in a human or animal subject by the method of the present invention can be effective for alleviating pain although amounts of any one or more biological response modifiers administered to a particular subject are at least one order of magnitude less than those amounts of biological response modifiers, such as TNF-α inhibitors or antagonists, that are provided to individuals who undergo systemic infusion or injection. By providing one or more biological response modifiers at or in close proximity to the site of inflammation or nerve damage, particularly when those biological response modifiers are provided in a controlled-release manner, the amount of biological response modifier that must be administered in relation to conventional modes of administration such as oral or by injection is decreased. This increases the pharmaceutical efficiency of the BRM, because it is being directed to the tissue in which its action will provide the greatest effect, such as a nerve root or region of the brain. While systemic delivery or delivery by injection may provide a sufficient level of therapeutic BRM to produce the desired result, it also results in an increased risk of unwanted side-effects, such as risk of infection when anti-TNFα compositions are repeatedly administered, thus resulting in increases in cost, inconvenience and discomfort to the patient.

Using the teaching within, effective dosages for use in the method of the present invention can be determined by those of skill in the art, particularly when effective systemic dosages are known for a particular BRM. Dosages may typically be decreased by at least 90% of the usual systemic dose if the BRM is provided as in the method of the present invention. In other embodiments, the dosage is at least 75%, at least 80% or at least 85% of the usual system dose for a given condition and patient population. Dosage is usually calculated to deliver a minimum amount of one or more BRMs per day, although daily administration is not required. If more than one pharmaceutical composition is administered, the interaction between the same is considered and the dosages calculated. Intrathecal dosage, for example, can comprise approximately ten percent of the standard oral dosage. Alternatively, an intrathecal dosage is in the range of about 10% to about 25% of the standard oral dosage. A protocol is provided herein for evaluating relative effectiveness and dosage requirements for newly-identified BRMs (especially TNF-α inhibitors) as compared to known compounds.

The controlled administration system of the invention can be positioned to deliver at the site of injury which is causing or will cause inflammation, such as a surgical site, or within about 0.5 to about 10 cm, or preferably less than 5 cm, for example, of the injury or inflammatory site. This site can comprise one or multiple sites in the spine, such as between the cervical, thoracic, or lumbar vertebrae, or can comprise one or multiple sites located within the immediate area of inflamed or injured joints such as the shoulder, hip, or other joints. In one embodiment, the controlled administration system is an implantable infusion pump which can be positioned elsewhere in the body, or externally to the body, and provided with one or more catheters to deliver BRMs to appropriate sites in the body. Implantation can occur simultaneously with surgery to repair a fracture, remove a tumor, etc., or can be performed in individuals who experience pain, especially chronic pain, as the result of earlier trauma, injury, surgery, or other initiation of inflammation.

“Localized” delivery is defined herein as non-systemic delivery wherein one or more BRMs are deposited within a tissue, for example, a nerve root of the nervous system or a region of the brain, or in close proximity (within about 10 cm, or preferably within about 5 cm, for example) thereto. “Controlled administration system” provides delivery of one or more BRMs in a quantity of pharmaceutical composition that can be deposited at the target site as needed for pain either continuously or at an intermittent rate.

In one embodiment, a controlled administration system comprises an interbody pump and a catheter, the catheter having a proximal end and a distal end, the proximal end having an opening to deliver a pharmaceutical composition in situ and a distal end of the catheter being fluidly connected to the interbody pump.

Timing of doses can also be determined by a physician or other appropriate health care professional, or the patient, based upon the condition, for example, severity and duration of pain. Duration of administration of BRMs, interval between doses, size of dose, continuity or spontaneity of dosage administration, are all appropriately determined by an individual's physician. In deciding the timing of doses the health care professional has options such as administering to a target site in a patient an effective amount of a pharmaceutical composition comprising one or more biological response modifiers, wherein the one or more biological response modifiers are administered by controlled administration system. The administration can (1) be localized and sustained, (2) occur over a period of at least one day to about 3 months, (3) be continuous or periodic. Further, the health care provider has the choice of selecting a pharmaceutical composition having a targeted release rate. For example, a targeted release rate is from about 24 hours to about 31 days. The health care provider may vary the combinations as the patient provides feedback over the treatment course. Accordingly, the health care provider has numerous options not previously available, especially in the treatment of pain, particularly chronic pain.

The method and system of the present invention has both human medical and veterinary use, being suitable for use in human children and adults, as well as in other mammals. Implantable controlled-delivery devices or compositions containing BRMs as described herein can be placed during orthopedic surgery to minimize inflammation and associated pain and to decrease the stimulus that often results in chronic pain, which becomes a disease state in itself.

In veterinary use, the controlled administration system and method of the invention can be useful for decreasing pain associated with orthopedic surgery or injury, or orthopedic or neurological damage associated with infection or inflammation. The method may be especially beneficial for larger animals such as horses, or smaller domestic pets such as cats and dogs.

For human medical use, the controlled administration system and method of the invention can be used to alleviate pain associated with rotator cuff injury or repair, articular joint pain or repair, temporomandibular joint disorder, tendonitis, rheumatoid and osteoarthritis, carpal tunnel syndrome, ligament pain or repair, or targeted muscular pain relief, for example. Examples of clinical indications for which the invention is appropriate include acute and chronic back and leg pain whatever the origin. In one embodiment, the BRM is delivered in the vicinity of an irritated nerve root at dose lower than current drugs. The BRM could be delivered over a period of a few days to several months depending upon the clinical indication. This directed and controlled delivery is beneficial as certain drugs, for example TNF-inhibitors, act to reduce the infection fighting capability of the immune system and therefore can lead to infection and other adverse events. Minimizing the amount of drug (in this case BRM) and targeting a site of delivery is a significant improvement over what is currently available. Further, the versatility of the treatment options, for example, modifying the dose and delivery at will, is unique. The health care provider can be more responsive to the patient feedback or changing clinical manifestations. Other inflammatory diseases may also be treated employing the invention. Biological response modifiers can be delivered singly, in combination, in series, or in simultaneously. One or multiple disc levels may be treated at the same time, with cervical, thoracic, lumbar, or multiple areas being targeted. Biological response modifiers may be applied interdiscally, adjacent to the disc, or intramuscularly. Biological response modifiers may be directed to inhibit the effects of TNF-α, cyclooxygenase 2, prostaglandin E2, mediators of inflammation such as glutamate, kinins such as bradykinin, and substance P, for example.

The invention is useful in the prevention and treatment of osteoporosis, osteoarthritis and rheumatoid arthritis. For example, rheumatoid arthritis, particularly, is known to have an inflammatory origin, and biological response modifiers such as inhibitors of the action of TNF-α can be useful, particularly when delivered by the implant and method of the present invention, for alleviating pain associated with these conditions.

Periprosthetic osteolysis is a major complication following total joint replacement. Articulating prosthetic joint surfaces and polymethylmethacrylate (PMMA) cement may generate wear particles that cause a chronic inflammatory response and osteoclastic bone resorption (wear debris-induced osteolysis), resulting in mechanical failure of the implant. TNF has been shown to mediate wear debris-induced, or wear particle-induced, osteolysis. Controlled and directed delivery of TNF inhibitors according to the controlled administration system and method of the present invention at an implant site provides a method for preventing implant-associated osteolysis. Osteolysis generally, whether wear-induced or caused by other factors, because it often occurs at individual sites such as sites of joint replacement surgery, is an appropriate target for therapy using the controlled administration system and method of the invention. Furthermore, because TNF-α has been found to induce osteoclast-like cells and the osteoclast is the cell that resorbs bone, sustained and directed (localized) administration of TNF-α inhibitors, particularly if combined with administration of osteoinductive factors such as BMP, LMP, or a combination of both, for example, can provide both pain relief and inhibition of bone resorption.

Similarly, malignant or benign tumors of bone are often associated with bone resorption. Where tumors are removed, partially removed, or where a tumor remains, there can be considerable pain. The method and system of the invention provides a means for alleviating such pain and making a cancer patient more comfortable, as well as inhibiting bone resorption or stimulating bone growth at the site.

In one embodiment, the method of the invention can be provided by a controlled administration system comprising an interbody or similar pharmaceutical pump, an optional catheter fluidly connected to the pump to provide a channel for at least one pharmaceutical composition to be transported from the pump to a target site, and a therapeutic quantity of at least one biological response modifier such as, for example, a TNF inhibitor. In one embodiment, such a system may also comprise at least one modified release pharmaceutical carrier for the at least one biological response modifier.

In an alternate embodiment, a depot can comprise at least one modified release pharmaceutical carrier for at least one biological response modifier, and a therapeutically effective amount of at least one biological response modifier, such as, for example, a TNF inhibitor. Controlled administration systems can be provided as kits, comprising at least one depot provided in sterile packaging and at least one aliquot of at least one biological response modifier in a package so that the biological response modifier is provided in sterile form when introduced into the body. Such kits can also comprise at least one package containing at least one aliquot of at least one biological response modifier in combination with one or more modified release pharmaceutical carriers. Kits can also provide modified release carriers containing biological response modifier within them, the modified release carriers being enclosed or partially enclosed within a matrix or containment device for complete or partial containment of the modified release carriers, the matrix or containment device being provided in sterile packaging and being appropriate for implantation into a target site within the body of a subject in need of therapy utilizing the at least one biological response modifier.

Methods, pharmaceutical compositions and biological response modifiers that are particularly effective for use in the method of the invention can be identified as shown in the following non-limiting examples.

### EXAMPLES

### Example 1

Evaluation of the effectiveness of protein-based inhibitors of TNFα function on mechanical injuries induced by sciatic nerve constriction (CCI) in rats, a model for investigation of chronic and acute pain syndromes, is performed to identify compounds having a significant pain inhibiting effect, and to establish optimal local dose levels of those compounds.

No. Animals

| | Systemic | Local | Local | Local |
|---|---|---|---|---|
| Treatment | dose | 10⁻¹ | 10⁻² | 10⁻³ |
| Vehicle only | 4 | | | |
| (neg ctrl) | | | | |
| Gabapentin | 4 | | | |
| (pos ctrl) | | | | |
| Compound #1 | 4 | 4 | 4 | 4 |
| Compound #2 | 4 | 4 | 4 | 4 |
| Compound #3 | 4 | 4 | 4 | 4 |
| TOTAL | 20 | 12 | 12 | 12 |

Four animals per group with CCI “neuropathic pain” are randomly assigned. Following administration of the test compound via systemic injection or a local delivery via an Alzet® osmotic pump, the CCI animals undergo a series of behavioral tests (i.e. mechanical Tactile allodynia and Thermal Nociceptive Test). The first dose is given prior to testing, with subsequent doses being provided at the half-life of each compound.
Behavioral Testing: Von Frey Test; Thermal Plate Test

Target compounds are delivered via local delivery through an osmotic pump, and behavioral testing for up to 8 times (four for each type of behavior), including the baseline, is performed. The length of study is 22 days or less. The systemic and local administrations, followed by behavioral testing as described below, are used to determine the optimal dosing regimen to be used with any proposed target compound that may be effective in the method of the invention.

The activity of compounds is evaluated using the in vivo Chronic Constriction Injury Model. A total of 56 Wistar (4/group) are recommended. CCI male rats weighing ˜300 g should be randomly assorted into treatment groups.

Thermal Paw Withdrawal Latency (PWL) (the Thermal “Nociceptive” Test) is assessed with a Thermal Analgesia Instrument on days 7, 14, and 21 and Mechanical Tactile Allodynia (Von Frey Filament Test) on days 8, 15, and 22. Preferably, each test is assessed for a maximum of 4 times each during the course of dosing including the baseline.

Assessment of Induced Chronic Neuropathic Pain by Chronic Constriction Injury (CCI) Surgery

Chronic constriction injury is generated on male rats. Under 2% isoflurane anesthesia, the animal's right common sciatic nerve is exposed and ligated by placing 3 loose ligatures using a method similar to that described by Bennet and Xie (1988). The common sciatic nerve is therefore exposed and freed from adherent tissue at mid-thigh by separating the muscle (biceps femoris) by blunt dissection. The loose ligatures are placed at 1 mm apart using chromic gut (4-0 absorbable suture). The Alzet® osmotic pump is implanted at this time in animals assigned to the localized delivery groups. The catheter tip of the pump is placed directly at the site of injury with the filled pump reservoir implanted subcutaneously on the back of the animal. A second surgery is performed at day 10 to exchange the TNF inhibitor-filled pump reservoir.

Behavioral Testing: Mechanical Tactile Allodynia: Von Frey Filament Test

Tactile allodynia is tested at the CCI ligated site as described in (Chaplan et al., J. Neurosci. Methods 53: 55-63, 1994). Briefly, the animals are placed in a clear plastic chamber with a wire-mesh bottom. Each animal is acclimated for 15 min prior to testing. Von Frey filaments (Stoelting, Wood Dale, Ill.) are used to determine the mechanical threshold for foot withdrawal (i.e., CCI site) by use of the up-down method of Dixon (Dixon, Annu. Rev. Pharmacol. Toxicol., 20: 441-462, 1980). The filaments, starting with one that possesses a buckling weight of 2.0 g and progressing up to one with a buckling weight of 15 g, are applied in sequence to the plantar surface of the right hind paw with a pressure that causes the filament to bend. Absence of a paw lifting/withdrawal response after 8 seconds prompts the use of the filament of the next higher weight. After an initial foot withdrawal response, the next larger filament is tested and the response noted. Four additional measurements are done using larger or smaller filaments depending upon the result of the previous measurement. The final five measurements are used to determine the foot withdrawal response score.

Thermal Paw Withdrawal Latency (PWL): Thermal Hyperalgesia Test

Thermal Paw withdrawal latency (PWL) is measured by thermal “nociceptive” stimuli response (hyperalgesia) using a plantar analgesia instrument (Stoelting Co, Wood Dale, U.S.A). Animals are placed on the plantar test apparatus clear plastic chamber, and allowed to acclimate for approximately 15 minutes (until the animal is at rest) prior to testing. Radiant heat light stimulus is applied to the CCI hind paw (right site) of each animal. The radiant heat source has an automated control-heat source timer, and paw withdrawal stops both heat source and timer. The heat source device preferably will be set at intensity 3 and a maximal cut-off of 20 sec should be set to prevent tissue damage.

### Example 2

Comparison and Ranking of Protein-Based Inhibitors of TNFα Function in the Chronic Constriction Injury (CCI) Rat Model: Systemic Versus Local Delivery

Systemic doses of compound are administered by subcutaneous injection starting the day of surgery, and periodically thereafter as determined by the half-life of the compound. Repeated injections of the compound should be given at the original dose level. Local administration of compound can be achieved by constant local infusion via an implanted osmotic pump.
Behavioral Testing: Von Frey Filament Test (Days 7, 14, and 21), Thermal Hyperalgesia Test (Days 8, 16, and 22)

Suggested experimental and control animal use numbers:

No. Animals

| Treatment Group | Systemic Dose | Local Dose |
|---|---|---|
| Vehicle (CCI Only) | 7 | 7 |
| Gabapentin (Pos control) | 7 | 7 |
| Compound 1 | 7 | 7 |
| Compound 2 | 7 | 7 |
| Compound 3 | 7 | 7 |
| TOTAL | 35 | 35 |

Blood is drawn (and can be taken from the retro-orbital plexus) at day 14 and at termination of the study. Blood is collected in EDTA tubes and stored at −20° C. Samples from all animals are collected for clinical pathology determinations.

At the completion of the comparative study (Day 22), sciatic nerve tissue is collected from all animals from each of the experimental and positive control groups. Animals are euthanized, preferably with an overdose of pentobarbitol, and sciatic nerves should be immediately removed, with a sufficient quantity being preserved in OCT compound and stored in a freezer at −70° C. for pathology staining/scoring.

Target compounds are effective for localized delivery in the method of the present invention if scores for those compounds that indicate inhibition of pain are equal to, or better than, the scores for known compounds used for systemic delivery, when the target compound is delivered at a dosage that is equal to, or preferably 10⁻¹ to 10⁻³ times, the systemic dosage.

### Example 3

Formulation of PLGA 50:50/rhBMP-2 Microspheres

Methylene chloride (Aldrich MO 02249E0, D=1.325) was used as a solvent. PLGA 50/50 was obtained from Sigma (Lactel BP-0100, lot 56H1176). Recombinant human bone morphogenetic protein (rhBMP) (7.31 mg/vial) was produced in the laboratory according to protocols previously described and known to those of skill in the art. Contents of 1 vial rhBMP were dissolved in 1 ml sterile water (preferably filter sterilized). PLGA (513.4 mg) was dissolved in 8 ml methylene chloride.

rhBMP was first dissolved in sterile water and the aqueous solution of BMP was then emulsified in the polymer solution of PLGA. Briefly, 0.5 ml of BMP solution, plus 4 ml of PLGA/MeCl₂ were combined and emulsified for 45 seconds using a homogenizer (medium setting). The emulsion mixture was transferred to a syringe having an 18 gauge needle. Sixty milliliters 3% PVA was added to a 150 ml glass beaker. The 3% PVA solution was stirred using homogenizer setting 3, and the emulsified polymer/BMP solution was added in dropwise fashion using the syringe and 18 gauge needle. After all polymer/protein was added, stirring continued at the same speed for 3-4 additional minutes. Stirred solution was then poured into a beaker containing 250-300 ml of sterile water and this solution was stirred for 2-3 hours using a magnetic stirrer, set at medium speed (5-6, generally). The solution was then vacuum filtered through a 0.22 micron filter. Two milliliters of sterile water was added and the spheres were stirred in the water. The spheres in water were transferred to a sterile polypropylene test tube, then frozen at −15° C for at least 3 hours before overnight lyophilization.

Formulation of PLGA 50:50/rhBMP-2 Microspheres Using Lyophilized BMP

Methylene chloride (Aldrich MO 02249E0, D=1.325) was used as a solvent. PLGA 50/50 was obtained from Sigma (Lactel BP-0100, lot 56H1176). Recombinant human bone morphogenetic protein (rhBMP) (7.6 mg/vial) was produced in the laboratory according to protocols previously described and known to those of skill in the art.

Briefly, 30.131 mg of lyophilized BMP-2 powder was added to 4 ml of PLGA/MeCl₂ and emulsified for 45 seconds using a homogenizer set at medium or mid-range. The emulsified mixture was transferred to a syringe fitted with an 18 gauge needle. Sixty milliliters of 3% PVA was poured into a 150 ml glass beaker. The PVA solution was stirred by homogenizer (setting 3) and emulsified polymer/BMP solution was added in dropwise fashion using the syringe and 18 gauge needle. After all polymer/protein was added, stirring was continued at the same speed for 3-4 more minutes. The solution was poured into a beaker containing 250-300 ml of sterile water and stirring continued for 2-3 hours using a magnetic stirrer (medium setting). The entire solution was then vacuum filtered through a 0.22 micron filter. The captured microspheres were rinsed 3 times with 4-5 ml of sterile water each rinse. Water was removed by vacuum filtration through a 0.22 micron filter, and approximately 2 ml of sterile water was added to the microspheres. Microspheres were stirred in the water, then transferred to a sterile polypropylene test tube. The microsphere solution was frozen at −15° C. for at least 3 hours before lyophilization overnight.

### Example 4

### PLGA-Enbrel™ Microsphere Preparation

Using the procedures described, microspheres were prepared. The procedure detailed below is used to make PLGA microspheres containing a protein (in this Example, etanercept is used, however other proteins are suitable) load of 10%. Depending on the encapsulation efficiency, the actual protein load will vary.

The materials include poly(DL-lactide-co-glycolide); 50/50 lactide/glycolide, ethyl acetate (reagent grade); polyvinyl alcohol (MW 40-70 k); sodium chloride (reagent grade); Enbrel™-etanercept (Lot D040637; 5 cc polypropylene syringes (silicone free); and sterile water.

Procedure

Applicants prepared 1 L of 1% (w/v) polyvinyl alcohol (PVA), 0.9% (w/v) NaCl solution using sterile water. Weighed and transferred 10 grams of PVA and 0.9 grams of NaCl to a 1 L glass beaker, then add 1 L of sterile water, then sterile filter the solution.

Applicants then prepared a 6.5% (w/w) solution of PLGA dissolved ethyl acetate. Obtaining an open vial containing the Enbrel™ formulation, and reconstitute the lyophilized cake with 0.3 mL sterile water. Transferring 3.6 mL of PLGA/ethyl acetate solution into an 8 mL vial, the Applicants then transferred the entire volume (0.3 mL) of reconstituted Enbrel™ to the vial containing the PLGA/ethyl acetate (1:12; aqueous:organic). Emulsifying the aqueous/organic mixture for 45 seconds using a handheld homogenizer, the Applicants then attached an 18 gauge needle to 5 cc syringe, and drew the homogenized emulsion into the syringe. Transferring 8 mL of 1% PVA solution to a beaker, the Applicants then steadily added the contents of the syringe dropwise to the PVA solution. After the entire contents of the syringe were expelled into the PVA solution, Applicants continued homogenizing for 40 seconds. An additional 8 mL of 1% PVA; 0.9% NaCl solution was added to the homogenized mixture and mixing was continued for 40 seconds. The mixture was decanted into a beaker containing 100 mL of 1% PVA; 0.9% NaCl solution and stirred on a magnetic stir plate on a medium setting for 4 minutes. Using a disposable pipet, 10 mL of the resulting suspension was transferred to each of two 15 mL polypropylene centrifuge tubes, each of which was centrifuged for 5 minutes. While using a pipet, the supernatant was removed from the tubes, then more of the suspended microspheres from the beaker was added and centrifuged again. This was repeated until the entire volume in the beaker was centrifuged. Afterwards the centrifuged microspheres were washed in 5 mL of sterile water (3×) and all the wash solutions were pooled. Thereafter, they were resuspended and the microspheres from the two tubes were combined. Finally the tube was frozen and the microspheres were lyophylized.

### Example 5

### In Vitro Elution of Enbrel™ Formulations

The following method was used to establish in vitro release profiles of Enbrel™ formulations.

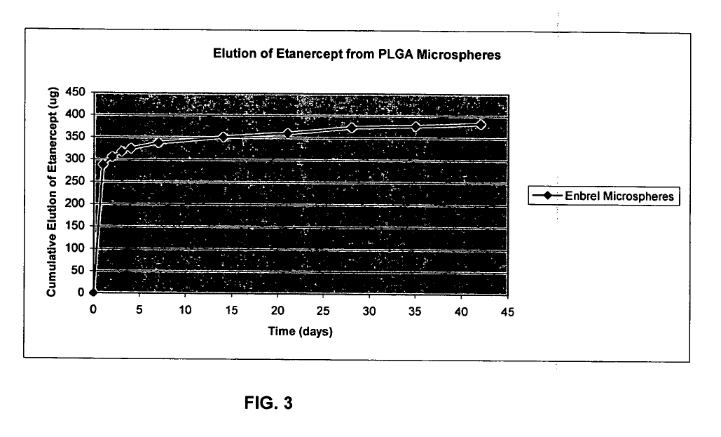

An exact amount of material (rod or microspheres) was weighed on an analytical balance. The Applicants transferred the material to a 4 mL glass vial and suspended the material in 2 mL of an appropriate buffer at physiological pH (7.4). the vial was capped and placed in an orbital incubator at 37° C. At selected time points, the buffer was replaced with fresh buffer. For samples containing microspheres, the tubes were first centrifuged to pull the solids down to the bottom of the tube, then the buffer was removed and replaced with an equal volume of fresh buffer. The vial was capped, labeled, and stored at 4° C. until analysis is done. (Samples containing rods were not centrifuged prior to replacing the buffer). The analysis of the exchanged buffer was done by HPLC and SDS-PAGE. FIG. 3 is a graph showing the elution results.

### Example 6

### PLGA-Enbrel™ Millicylinder Preparation

The materials include Poly(DL-lactide-co-glycolide); 50/50 lactide/glycolide; Acetone (reagent grade); Enbrel™-etanercept (Lot D040637); 3 cc Luer-Lok syringes (silicone free); 18 gauge stainless blunt tip dispensers; silicone tubing (0.045 in ID, 0.003 in wall); and binder clips.

The procedure detailed below is used for making solid polymeric (PLGA) rods containing a 5% (w/w) load of etanercept. The total formulation loading (including excipients) is approximately 15%.

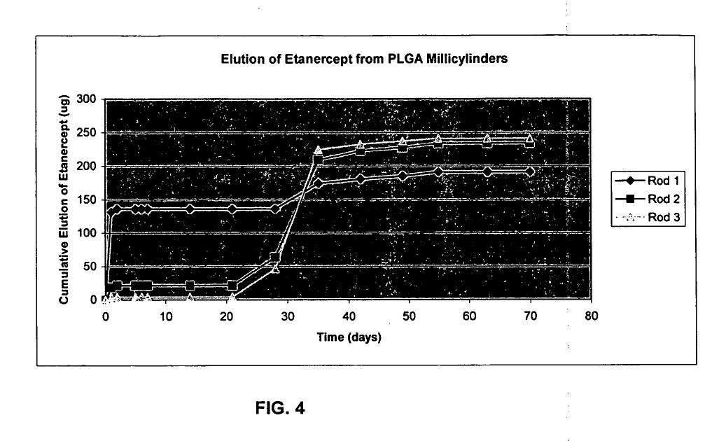

Applicants made a 40% (w/w) stock solution of PLGA in acetone by transferring 2 grams of PLGA to a small vial and bringing the total weight up to 5 grams with acetone. Next, they placed the mixture on an orbital shaker until the polymer was completely dissolved. Several segments of silicone tubing were cut to approximately 4 inches in length. A loose knot was tied in one end of each segment. An 18 gauge dispensing tip was attached to the other end of each tube segment, being sure the tubing slides at least 5 mm over the end of the dispenser tip. The vial containing the Enbrel™ formulation was opened and, using a small dry spatula, the lyophilized cake was broken up making sure that the contents of the vial exist as a free-flowing powder with no large clumps. The tip of a 3 cc syringe was placed into the polymer/acetone solution and approximately 1.5 cc of material was drawn into the barrel of the syringe. The vial containing the micronized Enbrel™ was placed on an analytical balance and the balance was tared. The Applicants dispensed approximately 1060 mg of PLGA/acetone from the syringe into the vial containing the Enbrel™ powder. Quickly thereafter, the viscous paste was mixed with a small spatula until the mixture appeared to be homogeneous, then the vial was capped to prevent evaporation of the solvent. Applicants then pulled a plunger out of a new 3 cc syringe, and transferred the mixed formulation from the vial to the back end of the syringe using a spatula. In most cases, complete transfer was not possible due to the high viscosity of the mixture. The plunger was replaced into the loaded syringe and pushed forward until all air is removed from the syringe. The syringe was attached to one of the previously prepared dispensing tips, assuring that the Luer fitting was secure between the syringe and the dispenser tip. Using one hand to hold the tubing over the dispensing tip, the formulation was pushed from the syringe into the tubing. When the formulation reached the loosely tied knot at the opposite end, the knot must be securely tightened. Applicants continued to push the formulation into the tubing until a bulge appeared in the tubing near the dispensing tip. Tubing was pulled from the dispenser tip, making sure that the bulged portion of the tubing was still present. While grasping the end of the tubing with one hand, a binder clip is secured to the end of the tubing with the other hand. The bulged section of the tube should be maintained through this procedure, as it is necessary to keep sufficient pressure within the tube, preventing collapse of the tubing. The above steps are repeated until all formulation from the syringe has been dispensed into the sections of silicone tubing. Leaving the sections of tubing at room temperature for 24 hours, they were allowed to dry under vacuum at room temperature for another 24 hours. After vacuum drying, the silicone tubing was removed from the hardened rods by gently slicing lengthwise along each rod using a scalpel. The tubing was peeled off the rods using a pair of forceps. The Applicants recorded weights for each rod and placed them under vacuum for another 24 hours at room temperature. The rods were weighed again to assure that all solvent has been removed. The rods were placed in a tightly sealed vial, and a strip of Parafilm was placed around the cap. The rods are stored at 4° C. until needed. FIG. 4 is a graph of the elution results.

### Example 7

### TNF Inhibitor Implant

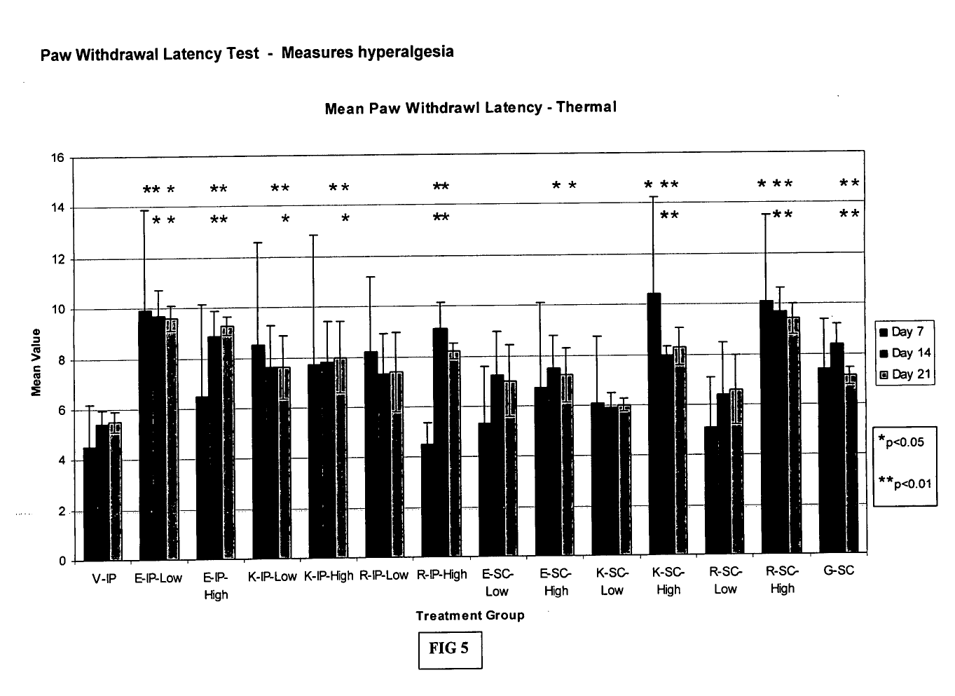

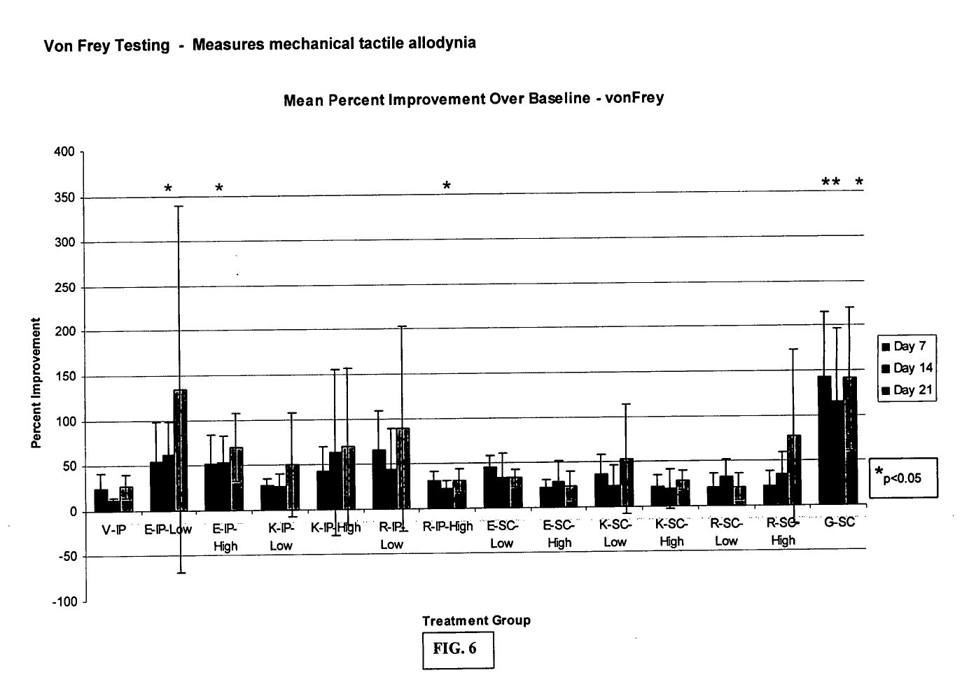

Using selected inflammatory cytokine inhibitors, Applicants conducted sciatic nerve constriction injury (CCI) rat model studies to compare the cytokine inhibitors. CCI rat model studies to establish local effect vs. systemic effect (3 compounds). Surgeries were performed for a 4-week dosing study to be followed up with a 6-week study comparing systemic injection versus local delivery via implanted pump. FIGS. 5 and 6 are graphical representations of the results of the Paw Withdrawal Latency test which measure hyperalgesia (FIG. 5) as well as the Von Frey Testing which measures tactile allodynia (FIG. 6).

In this CCI Rat Model Comparison Study, three loose ligatures were placed around animals right common sciatic nerve. At Days 7, 14 and 21, the von Frey Filament test was performed. On Days 8, 15 and 22, the Thermal Paw withdrawal latency test (Days 8, 15, 22). At Day 22, the subject animals were sacrificed. Below is a table disclosing the compound used, the route of administration, the frequency of administration, the dose(s) and any relevant comments.

Compound Dosing (n = 4)

| | Route of | Frequency of | | | | | |
|---|---|---|---|---|---|---|---|
| Compound | Administration | Administration | Dose 1 | | Dose 2 | | Comments |
| Vehicle | IP | Every 3 days | — | | n/a | | Injury Only |
| treatment (PBS) | | | | | | | |
| Gabapentin | SC | 1 hour prior to | | | n/a | | Positive Control |
| | | behavioral tests | | | | | |
| Enbrel | IP | Every 3 days | 2.4 | mg/kg | 8 | mg/kg | Test Compound |
| Enbrel | SC | Every 3 days | 2.4 | mg/kg | 8 | mg/kg | Test Compound |
| Remicade | IP | Every 8 days | 2.4 | mg/kg | 8 | mg/kg | Test Compound |
| Remicade | SC | Every 8 days | 2.4 | mg/kg | 8 | mg/kg | Test Compound |
| Kineret | IP | Every day | 1 | mg/kg | 10 | mg/kg | Test Compound |
| Kineret | SC | Every day | 1 | mg/kg | 10 | mg/kg | Test Compound |

### Example 8

Evaluating the Local Delivery of Selected Protein-Based Inhibitors of TNFα and IL-1β Function on Sciatic Nerve Constriction Injury: Model of Chronic Neuropathic Pain.

In this Example, Applicants establish the efficacy of low dose, local application of two compounds on mechanical injuries induced by sciatic nerve constriction injury. Rats with chronic constriction injury (CCI) of the sciatic nerve are used in these studies. Based on previous data, Applicants selected the Low IP dose to repeat that group in this Example. Dose 1 through the Alzet pump is equal to the IP dose; Dose 2 is a 10-fold decrease.

Compound Dosing (n = 6)

| | Route of | Frequency | | | | |
|---|---|---|---|---|---|---|
| | Admini- | of Admini- | | | | |
| Compound | stration | stration | Dose1 | | Dose 2 | |
| Vehicle | Alzet | — | — | | — | |
| treatment | Pump* | | | | | |
| Gabapentin | SC | 1 hour prior | — | | — | |
| | | to behavioral | | | | |
| | | tests | | | | |
| Enbrel | IP | Every 3 days | 2.4 | mg/kg | — | |
| Enbrel | Alzet | — | 10.0 | μg/hr | 1.0 | μg/hr |
| | Pump* | | | | | |
| Remicade | IP | Every 8 days | 2.4 | mg/kg | | |
| Remicade | Alzet | — | 3.75 | μg/hr | 0.375 | μg/hr |
| | Pump* | | | | | |
| Kineret | IP | Every day | 1 | mg/kg | | |
| Kineret | Alzet | — | 12.5 | μg/hr | 1.25 | μg/hr |
| | Pump* | | | | | |

*Pump reservoirs are exchanged on Day 10.

Behavioral testing is conducted: The behavioral tests are the von Frey filament test (mechanical tactile allodynia) on Days 7, 14, and 21, and the thermal paw withdrawal test (thermal nociceptive test using a thermal analgesia instrument) on Days 8, 15, and 22.

1. A method for reducing pain, the method comprising administering to a target site in a subject in need of treatment an effective amount of a pharmaceutical composition comprising one or more biological response modifiers, wherein the one or more biological response modifiers are administered by a controlled administration system.

2-5. (canceled)

6. The method of claim 1, wherein the pharmaceutical composition has a targeted release rate.

7. The method of claim 6, wherein the targeted release rate is from about 24 hours to about 31 days.

8. The method of claim 6, wherein the targeted release rate is from about at least one day to about three months.

9. The method of claim 1, wherein the controlled administration system is implanted in a subject at or near a target site.

10. The method of claim 9, wherein the target site is an inflamed nerve.

11. The method of claim 9, wherein the target site is a spinal site.

12. The method of claim 10, wherein the spinal site is a spinal disc or an intervertebral space.

13-22. (canceled)

23. The method of claim 1, wherein the biological response modifier is selected from the group consisting of soluble tumor necrosis factor α receptors, pegylated soluble tumor necrosis factor α receptors, monoclonal antibodies, polyclonal antibodies, antibody fragments, COX-2 inhibitors, metalloprotease inhibitors, glutamate antagonists, glial cell derived neurotrophic factors, B₂ receptor antagonists, Substance P receptor (NK1) antagonists, Downstream regulatory element antagonistic modulator (DREAM), iNOS, inhibitors of tetrodotoxin (TTX)-resistant Na⁺-channel receptor subtypes PN3 and SNS2, inhibitors of interleukins, TNF binding protein, dominant-negative TNF variants, Nanobodies™, kinase inhibitors, and combinations thereof.

24. The method of claim 1, wherein the biological response modifier is selected from the group consisting of Adalimumab, Infliximab, Etanercept, Pegsunercept (PEG sTNF-R1), Onercept, Kineret®, sTNF-R1, CDP-870, CDP-571, CNI-1493, RDP58, ISIS 104838, 1→3-β-D-glucans, Lenercept, PEG-sTNFRII Fc Mutein, D2E7, Afelimomab, AMG 108, 6-methoxy-2-napthylacetic acid) or betamethasone, capsaiein, civanide, TNFRc, ISIS2302 and GI 129471, integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL6 mAb (MRA, Tocilizumab, Chugai), HMGB-1 mAb (Critical Therapeutics Inc.), anti-IL2R antibody (daclizumab, basilicimab), ABX (anti IL-8 antibody), recombinant human IL-10, HuMax IL-15 (anti-IL 15 antibody) and combinations thereof.

25-26. (canceled)

27. An implant comprising a pharmaceutical composition comprising one or more biopolymers and at least one biological response modifier.

28. The implant of claim 27, wherein the biopolymers are chosen from the group consisting of poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, polyphosphoesters, polyanhydrides, polyester-anhydrides, polyamino acids, polyurethane-esters, polyphosphazines, polycaprolactones, polytrimethylene carbonates, polydioxanones, polyamide-esters, polyketals, polyacetals, glycosaminoglycans, hyaluronic acid, hyaluronic acid esters, polyethylene-vinyl acetates, silicones, polyurethanes, polypropylene fumarates, polydesaminotyrosine carbonates, polydesaminotyrosine arylates, polydesaminotyrosine ester carbonates, polydesamnotyrosine ester arylates, polyethylene oxides, polyorthocarbonates, polycarbonates, or copolymers or physical blends thereof or combinations thereof.

29-33. (canceled)

34. A method for treating osteolysis and/or bone resorption comprising administering to an osteolytic site in a subject in need of treatment an effective amount of a pharmaceutical composition comprising one or more biological response modifiers, wherein administration of the pharmaceutical composition is localized and sustained.

35. The method of claim 34, wherein the one or more biological response modifiers is administered in conjunction with at least one osteoinductive factor.

36. The method of claim 35, wherein the osteoinductive factor is a bone morphogenetic protein, a biologically active bone morphogenetic protein fragment or variant, a LIM mineralization protein, a biologically active LIM mineralization protein fragment or variant, or a combination thereof.

37. A method for alleviating pain associated with bone tumors, the method comprising administering to a tumor site in a subject in need of treatment an effective amount of a composition comprising one or more biological response modifiers, wherein administration of the composition is localized and sustained.

38. The method of claim 37, wherein the one or more biological response modifiers is administered in conjunction with at least one osteoinductive factor.

39. The method of claim 38, wherein the osteoinductive factor is a bone morphogenetic protein, a biologically active bone morphogenetic protein fragment or variant, a LIM mineralization protein, a biologically active LIM mineralization protein fragment or variant, or a combination thereof.

40. A system for providing pain relief medication in a mammalian subject, the system comprising controlled administration system for providing controlled and directed delivery of at least one biological response modifier to a target site in a subject in need thereof comprising an effective amount of a composition comprising at least one biological response modifier which decreases inflammation at the target site.

41-45. (canceled)

46. The system of claim 40, wherein the controlled administration system comprises a catheter having a proximal end and a distal end, the proximal end having an opening to deliver a pharmaceutical in situ, the distal end being fluidly connected to a pharmaceutical pump.

47. The system of claim 46, wherein the proximal end of the catheter delivers the biological response modifier within about 1 mm to about 10 cm of the target site.

48. The system of claim 46, wherein the proximal end of the catheter delivers the biological response modifier within a range of about 1 cm to about 5 cm of the target site.

49-51. (canceled)

52. The system of claim 40 further comprising a therapeutically effective amount of at least one osteoinductive factor.

53. The system of claim 52, wherein the osteoinductive factor comprises a bone morphogenetic protein, a biologically active bone morphogenetic protein fragment or variant, a LIM mineralization protein, a biologically active LIM mineralization protein fragment or variant, or a combination thereof.

54-62. (canceled)

63. The method of claim 1, wherein the BRM is a COX-2 inhibitor.

64. The method of claim 63, wherein the BRM is 6-methoxy-2-napthylacetic acid) or betamethasone.

65. The method of claim 1, wherein the BRM is a metalloprotease inhibitor.

66. The method of claim 65, wherein the metalloprotease inhibitor is TAPI.

67. The method of claim 1, wherein the BRM is selected from the group consisting of glutamate antagonists, glial cell-derived neurotropic factors (GDNF), B₂ receptor antagonists, Substance P receptor (NK1) antagonists, Downstream regulatory element antagonistic modulator (DREAM), iNOS, inhibitors of tetrodotoxin (TTX)-resistant Na⁺-channel receptor subtypes PN3 and SNS2, inhibitors of interleukins.

68. The method of claim 67, wherein the Substance P receptor (NK1) antagonist is capsaicin or civanide.

69. The method of claim 67, wherein the inhibitor of interleukin is selected from the group consisting of IL-1, IL-6 IL-8, and IL-10.

70. The method of claim 1, wherein the BRM is a TNF binding protein.

71. The method of claim 70, wherein the TNF binding protein is Onercept.

72. The method of claim 1, wherein the BRM is an inhibitor of an interleukin.

73. The method of claim 72, wherein the interleukin is IL-1, Il-6, IL-8, or IL-10.

74. The method of claim 1, wherein the BRM is a kinase inhibitor.

75. The method of claim 74, wherein the kinase inhbitor is selected from the group consisting of Gleevec, Herceptin, Iressa, imatinib (STI571), herbimycin A, tyrphostin 47, erbstatin, genistein, staurosporine, PD98059, SB203580, CNI-1493, VX-50/702, SB203580, BIRB 796, Glaxo P38 MAP Kinase inhibitor, RWJ67657, UO126, Gd, SCIO-469, RO3201195, and Semipimod.

76. The method of claim 1, wherein the BRM is ISIS2302 and GI 129471.

77. The method of claim 1, wherein the BRM is selected from the group consisting of integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, Tocilizumab, Chugai), HMGB-1 mAb (Critical Therapeutics Inc.), anti-IL2R antibody (daclizumab, basilicimab), ABX (anti IL-8 antibody), recombinant human IL-10, HuMax IL-15 (anti-IL 15 antibody).

78. A method for retarding tissue necrosis and/or damage, the method comprising administering to a target site in a subject in need of treatment an effective amount of a pharmaceutical composition comprising one or more biological response modifiers, wherein the one or more biological response modifiers are administered by controlled administration system.

79. The method of claim 78, wherein the administration is localized and sustained.

80. The method of claim 78, wherein the controlled administration system is implanted in a subject at or near a target site.

81. The method of claim 78, wherein the target site is an inflamed nerve.

82. The method of claim 78, wherein the target site is a spinal site.

83. The method of claim 78, wherein the spinal site is a spinal disc or an intervertebral space.
